(19) United States
(12) Patent Application Publication
(10) Pub. No.: US 2007/0212367 A1
(43) Pub. Date: Sep. 13, 2007

(54) Novel immunologically active peptide fragments of a proline-rich polypeptide isolated from colostral mammalian fluids for treatment of viral and non-viral diseases or diseased conditions

(76) Inventor: Andrew Maurice Keech, Phoenix, AZ (US)

Correspondence Address: DR. ANDREW MAURICE KEECH, 5132 N. 31st WAY, # 127, PHOENIX, AZ, 85016, US

(21) Appl. No.: 11/319,936

(22) Filed: Mar. 13, 2006

(51) Int. Cl. A61K 39/00; A61K 35/20

(52) U.S. Cl. 424/185.1; 424/535

(57) ABSTRACT

Present invention relates to an immunologically active nonapeptide fragments of a proline-rich polypeptide isolated from mammalian colostral fluids for treatment of viral and non-viral diseases or diseased conditions, a method and a system for processing mammalian colostral fluids for isolating target peptides and proteins, a pharmaceutical formulation containing such peptides and its use for the treatment of viral diseases or diseased conditions. The product comprises proline-rich polypeptide of immuno regulatory properties. The method of processing includes, steps of passing bodies fluids, such as, mammalian colostral fluids through at least one ion exchange column such as an anionic resin and/or a cationic resin. The ion exchange column can be selected to retained large particles at a first pH level selected to remove such large particles at a maximum fluid pressure of 30 pounds per square inch (psi), preferably less than 10 psi.

### FIELD OF INVENTION

Present invention relates to nutraceutical and its composition for treatment of viral and non-viral diseases or diseased conditions. It more particularly relates to Proline-Rich Polypeptide (PRP) isolated from mammalian colostral fluids and its pharmaceutical composition for the treatment of the viral and non-viral diseases or diseased conditions. Further, the invention also relates to method of isolating and preparing said PRP from mammalian colostral fluids.

### DESCRIPTION OF THE PRIOR ART

Since the early 1970's whey proteins have been concentrated for uses in healthcare and for use in nutritional and functional foods, for example as an ingredient that are now a days termed as “nutraceuticals”. These polypeptides include beta-lactoglobulin and alpha-lactalbumin that are commonly extracted from bovine milk. More recently, lactoferrin and immunoglobulins also have been extracted from bovine milk, but to a lesser extent. Although the concentrations of lactoferrin (100-200 mg/liter) and the immunoglobulins (5-6% of total whey proteins) are low in milk, the concentrations of lactoferrin (1-2 g/liter), proline-rich polypeptides and immunoglobulins (70-80% of total whey proteins) are much higher in mammalian colostral fluids.

Some protein functions involve the binding to other molecules called ligands. Ligands can be drugs, hormones or antigens, which can bind with other proteins. Those compounds that can act as ligands but which normally are found naturally in animal bodies fall into three general classes: neurotransmitters, steroids (including the sex hormones), and peptides. The first two classes are considered to be bioactive, whereas peptides are not. (Bioactive is a phrase that describes a set of compounds which have an effect on animal cells that is in direct proportion to their number and which are produced either outside the body of the animal or only in specialized organs or systems thereof.) Peptides or polypeptides typically have a relatively low molecular weight and are the product of the sequential covalent bonding of several amino acids during protein synthesis. For example, peptides typically are formed from about 4 to 100 sequentially bonded amino acids.

Proline-rich-peptides and/or non-ionic peptides play key role in various biochemical processes occurring in healthy as well as diseased animals including human being. These peptides may be receptor peptides and are in fact very specialized types of signal proteins. They reside in or on the exterior surfaces of all animal cells (regardless of particular cellular function assigned). When activated through interaction with a ligand, a receptor then transmits a biochemical message into the interior of the cell through cascade of reaction(s).

Peptides, such as proline-rich-peptides and non-ionic peptides, which function as ligands are produced in the ribosomes of all or very nearly all of the cells of animals including human being. They are sometimes referred to as informational peptides because they contain specific information to trigger specific biological processes. The informational peptides also may help to protect the cells by re-orientating receptor sites often used by synthetic viral protein ligands. Thus, these peptides help to inhibit viruses from attaching themselves to those specific target cells by regulating immuno-modular and cytokine intercellular function and intracellular function.

These peptides are relatively small, at least in relation to most proteins and having molecular weights of at least 20,000 Daltons. In general, peptides have a molecular weight not more than about 1000 Daltons, although some are larger or even larger as about 6000 Daltons. Nevertheless, they are significantly smaller than the most of the proteins occurring in the body of the mammals.

When a peptide leaves the cell in which it is produced, it moves throughout the body by way of the body fluids, for example, interstitial body fluids between the cells and the circulatory system. In the blood and interstitial fluids, peptides tend not to agglomerate with themselves (i.e., they remain separate). This separateness allows these peptides to remain in forms in which they bind to appropriate receptors. For instance, a peptide produced by one cell can be transported to and interact with cellular function of a distant cell. When such an interaction occurs, a type of biochemical transmission to the cell interior is set into motion through as cascade of changes and in turn, induces some type of a response within the cell. One such cellular action is believed to be the production of additional peptides of the same type.

Some viruses take up residence in animal bodies by entering cells through particular types of receptors. If the necessary type of receptor already is bound to another ligand, such as a peptide, or the shape of the receptor does not or is no longer compatible with the viral ligand, then the virus cannot enter that given cell and must find another cell in which to enter. If all cells have the target receptor bound with other ligands, or there has been a conformational change of shape at the receptor site because of biochemical processes from within the cell, the virus' entry path is blocked and infection is averted.

When an animal, including a human, is healthy, it has a full or very nearly full complement of such a ligand like peptides. However, due to any one or more of a variety of factors, such as increased age of the animal, bodily abuse by environment or substance abuse, nutrition, suppressed immune system, and/or illnesses and diseases, an animal may fail to produce or maintain one or more of these types of peptides. Such failures often can be the first cause of illness or diseased condition. Return to health can be relatively quick and easy, however, when the missing peptide(s) is reintroduced into the body because such peptides can, as described above, “instruct” cells to create more copies of the peptides. These are commonly called “proline-rich-polypeptides” (PRPs), “cytokine precursors” or “immuno-modulating peptides”. Commonly, these peptides have been called the “software of the cell” or “software of the human operating system”, which refer to the information required for all living mammalian cells to function. The initialization of correct cellular function is started when a female lactating mammal first delivers the colostral milk to a newborn mammal baby that is commonly known as “passive immunity”. In addition to such passives immunity, the colostral fluids also provide cytokine precursors to initiate many biochemical processes in mammalian cells. Thus, reintroduction of a small amount—perhaps a single copy—of one or more missing peptides to any infant, teenage, adult or elderly human, or any aged mammal including human being, can quickly return cells in the body to their normal amount of the peptide(s) in question.

The target peptides can be derived from blood or from other mammalian bodily fluids derived from or in contact with blood. Such fluids include, but are not limited to, milk, colostral fluids, semen, urine, vaginal fluid, and the like. However, in materials such as milk and colostral fluids, for example, peptides should be in what is called essentially an impaired state because they may be agglomerated with or on much larger biochemical macromolecules i.e. fats or other proteins. Additionally, ingestion by eating or drinking certainly denatures the peptides because of the acidic conditions of the stomach and the relatively aggressive enzymatic actions in the intestine. Thus, although many external sources of peptide ligands are available, these peptides are often in a form that renders them useless for the desired effect. Accordingly, processing or refinement of such external sources is necessary to obtain these small amount short peptides in purified form for reintroduction to any infant, teenage, adult or elderly human, or any aged mammal including human being.

Of the external sources of such peptides, the one that seems to provide them in the highest concentrations and is most widely available is colostral fluids. This material has been the subject of numerous processing methodologies. However, almost all of the previously described processing methods appear to have been directed at collecting or isolating biologically active macromolecules that are much larger than rich peptides, such as, for example, proteins, lactoferrin, immunoglobulin, lipids, etc.

In the realm of inductive science, the dominant paradigm can seldom be challenged in a frontal attack, especially when it is apparently successful, and only what Kuhn calls ‘scientific revolutions’ can overthrow it. Thus, it is hardly surprising that the concept of these proline-rich peptides is considered with contempt since its putative mode of action contravenes dogmas of both immunology and molecular biology. Because of the failure of medical science to manage the AIDS pandemic, peptides that has been successfully used for treating or preventing viral infections, may today overcome a priori prejudice and rejection more swiftly.

Emerging strains of new, antibiotic-resistant “super-bugs” are a global problem. Over a dozen new food borne pathogens have been identified in the last twenty years. Just as clear evidence suggested a solution in dealing with the Black Plague, so too is clear evidence indicates a potential solution to our modern plagues. We must take individual responsibility for our own health by strengthening our immune systems. This is the most critical health issue we face and these proline-rich peptides can play a major role in maintaining our immediate and long-term health.

Importantly, the present invention provides proline-rich polypeptide isolated from mammalian colostral fluids for treatment of viral diseases as function of immuno regulatory properties. Also, provided the method of producing proline-rich polypeptide form colostral fluids processing and system for producing the same to encourage relatively high fluid pressures and much lower yields of peptides as a side effect of fast processing speeds and current technologies used. Moreover, the method of treatment and use of therapeutical formulation are also provided.

Accordingly, there remains a need for a proline-rich polypeptide and a method and system for producing the such peptides by processing mammalian colostral fluids that result in an end product which is peptide-rich, with the same efficacy as in its native state, but substantially free from other materials that can denature such peptides, and thus able to fully express their peptide bioactivity without steric hindrance, and increase liquid diffusion of these peptides for the treatment of fatal viral diseases of animals including human being. Similarly, also remains the need of a method for treatment of the diseases or diseased condition due to viral infections.

### DESCRIPTION OF THE INVENTION

The present invention relates to an immunologically active peptide fragment of a proline-rich polypeptide from mammalian colostral fluids having immuno-regulatory properties. These peptides include short chain proteins with a high concentration of the amino acid Proline. These bioactive peptides support regulation of thymus gland that is responsible for the normal development of immunological function in the body. These are mainly characterized by PRP1, PRP2, PRP3, and to a lesser extent PRP4 and PRP5.

This Proline-Rich Polypeptide (PRP) is actually a hormone that regulates the thymus gland, which is responsible for stimulating an under active immune system or down-regulating an overactive immune system as seen in autoimmune disease. PRP inhibits the overproduction of lymphocytes and T-cells and reduces the major symptoms of allergies and autoimmune disease such as pain, swelling and inflammation. It regulates activity of the immune system as hormones of the thymus gland. It activates an under active immune system, thereby helping it to move into action against disease-causing organisms.

PRP also suppresses an overactive immune system, such as is often seen in the autoimmune diseases. It is highly anti-inflammatory and acts on T-cell precursors to produce helper T-cells and suppresser T-cells that turns white blood cells into functionally active T cells.

PRP contains tiny protein molecules produced by immune T-cells that play a central part in promoting immune system strength and effectiveness. It contains a set of messaging molecules that convey immune information within an individual's immune system. Nature also uses PRP to carry immune information from one individual's immune system to another individual. These are the signal proteins that transfer immunity from one person to another, which is vital to the sound health.

PRP is small immune messenger molecule that transfers immune recognition signals between immune cells and thereby assists in educating naive immune cells about a present or potential danger. These peptides are messengers, passing immunity information about the presence of an immune threat—whether external or internal—and how to properly respond, from immune cell to immune cell.

It is small peptides of about eight amino acid residues. Eighteen different amino acids are reported, which after re-introduction in the animal or human being, after combination create billions of different transfer factors such as PRP.

A component of the immune system is the natural killer (NK) cell. The function of NK cells is extremely important in protecting against the development or progression of any infection, including HIV. In determining treatment recommendations for AIDS, the CD4 count and viral load are routinely used. However, for a more complete assessment of overall immune function, a measure of NK cell function is also helpful.

Unlike other immune cells that must first obtain information from CD4 helper cells, the NK cell can target and kill antigens both inside and outside of cells without “conferencing” with other immune cells. Acting on their own volition, NK cells can target and kill viruses, cancer cells, bacteria, and many other antigens. However, NK cell function can be impaired by the failure of some infected cells to present antigens on their cell surfaces, which signal the NK cell. This disables the NK cell, causing it to not “see” the infected cell directly, thereby diminishing its ability to destroy it. Multitest CMI is especially valuable in this situation when used with an NK function test. A high NK function score of 100 or more lytic units indicates that the NK cells can “see” the infected cells and have the energy to destroy them. A high lytic unit value implies correct NK function, but coupled with a low Multitest CMI score or anergy, would still indicate immune dysfunction because diminished antigen presentation will blind the NK cells from “seeing” all infected cells that need to be destroyed.

In this way, both tests are used synergistically for a broader evaluation of immune function. Multitest CMI and NK function tests may be considered to be the polygraph test for the efficacy of all AIDS treatment protocols. High CD4 counts and low PCR viral loads are used as immune markers, but they are not always indicators of actual immune function. High CD4 counts and low PCR viral loads without correct immune functioning will not protect you from PCP, CMV, TB, or other opportunistic infections (OI). Actual measurable improvements of immune function may, even with low CD4 cells and high PCR viral loads, offer protection from OI.

Proline-rich Polypeptides show an increase in Lytic Activity after just a few days of oral ingestion: In a population of 107 participants (59 females, 48 males, average age 53 years ranging from 17 thru 83) initial killer cell activity was 18 Lytic Units (a measure of killer cell activity) and the final killer cell activity was 246 Lytic Units, after 1 week. A lytic unit is a measured by microscopically observing the action of 1 Natural Killer Cell. A reading of 250 indicates this one NK cell kills 250 cancer cells under a controlled laboratory conditions.

By comparison, the average NK cell activity in the U.S. population is 20-50, whereas in a healthy population it is 150-225.

Accompanying this dramatic increase in Natural Killer T cell activity were vast improvements in the population of 107 consisting of 50% cancer patients, 30% chronic fatigue syndrome, and the remaining 20% a mixed bag of lupus, allergies, fibro myalgia, blood disorders, hepatitis C, colitis, chronic infections, recurrent infections, autoimmune diseases, and cervical dysplasia/metaplasia.

Average time of treatment using this Proline-rich Polypeptide Colostral Extract is 6-12 months to restore normal homeostasis in humans.

PRP directly supports the Natural Killer (NK) cells of the immune system. Natural Killer Cells provide the front line of defense specially equipped to locate and kill disease cells. NK cells attach to the surfaces of foreign substances or their outer cell wall, and inject a chemical “grenade” (granule) into the interior. Once inside, the granules explode and destroy the foreign invader within five minutes. The NK cell itself remains intact and moves on to destroy the next immune attacker. Strong, active and optimally functioning NK cells promote optimal health and deter foreign substances from affecting immune function.

Importantly PRP does not elicit an allergic response and is not species-specific. This means the peptides produced using colostral fluids of a cow are as effective in humans as they would be in another cow. This exciting ability could spark a revolution in medicine and has prompted the inventors to use successfully these peptides for the treatment of viral infection and autoimmune disorders.

Isolated peptide of the present invention is a pure, natural product made from mammalian milk colostral fluids. It is a no fat liquid that contains >95% colostral fluid peptides on a protein basis. It is processed at low temperatures to maintain maximum activity and is produced in a sterile environment. The colostral fluids used in the present invention for isolating the PRP is free from hormone, pesticide and antibiotic.

The final product of the present invention contains protein (nitrogen×6.38)—0.3-0.4%, lactose—3.0-4.0%, pH—4.2-4.9, fat—0.0% and minerals—<0.3%.

Microbiological analysis reveals that it has standard plate count (cfu/g)—<10, coliform (cfu/g)—negative; Staphylococcus aureus—negative, yeast and mold (cfu/g)—<1 and pathogen—negative.

Physical properties of the product of the present invention are appearance—clear green liquid, flavor—slightly acidic and natural cream flavor and shelf life is 365 days in dry, aseptic and refrigerated environmental condition.

The molecular weight of the peptides determined by gel filtration is 17 200 Daltons. However, in the presence of guanidinium chloride the molecular weight found is about 6000 Daltons. The polypeptide contains about 22% of proline, a high proportion of non-polar amino acids, a low percentage of glycine, and no alanine, arginine and cysteine residues. The only N-terminal amino acid found is leucine. C.D. spectra in water and in 50% (v/v) trifluoroethanol suggest the presence of block sequences of proline residues forming helices of polyproline II type. The proline-rich polypeptide is soluble at 4° C. but is reversibly precipitated on warming to room temperature. Maximal precipitation is observed at pH 4.6 and at ionic strength above 0.6. The precipitation depends on the concentration of the polypeptide. No effect of other proteins, Ca2+ and Zn2+ ions on the precipitation of the polypeptide was found. The proline-rich polypeptide is not an amphipathic protein. The lack of effect of the polypeptide on proteolytic enzymes ruled out the possibility that it is an inhibitor of proteinases.

According to another aspect of the present invention, it relates to a pharmaceutical formulation comprises an immunologically active peptide fragments of a proline-rich polypeptide isolated from mammalian colostral fluids and pharmaceutically acceptable carriers. The formulation of the present invention is in the form of spray for oral administration of PRP in an effective amount so that can be held or circulated in mouth for dew seconds before swallowing.

In another embodiment of the present invention, the formulation of the present invention can be directly administered in an effective amount as a teaspoon of liquid in the mouth so that it can be held in contact with oral mucosa for a few minutes and then swallowed.

In still another embodiment of the present invention, the formulation of the present invention can be directly administered in mouth in an effective amount, for example ¼ to 1 teaspoon.

Alternatively, the formulation of the present invention can be topically used or applied on burns and sutures, and used for skin healing conditions.

According to another object of the invention, the invention relates to a method for treatment of viral and non-viral diseases or diseased conditions comprises administrating orally pharmaceutically effective amount of formulation of the present invention, comprising an immunologically active peptide fragments of a proline-rich polypeptide isolated from mammalian colostral fluids.

As per preferred embodiment of the present invention, the formulation of the present invention can be used effectively to treat a wide range of diseases or diseased conditions which include, but not limited bacterial, mycobacterial, fungal, parasitic, viral diseases or diseased conditions.

According to further embodiment of the present invention, the formulation of the present invention can be administered as an oral delivery spray or directly held in the mouth for not less than 30 seconds as a teaspoon of dose to allow sorption through the mucous membrane of the mouth.

According to specific embodiment, for adults with non-viral infections, the formulation of the present invention can be typically administered twice a day, wherein each administration consists of five successive 1 ml sprays. For children, the formulation of the present invention can be preferably administered twice a day, wherein each administration consists of two or three 1 ml sprays. For infants, the formulation of the present invention can be preferably administered twice a day, wherein each administration consists of one 1 ml spray.

According to another specific embodiment, for adults with viral infections, the formulation of the present invention can be typically administered every four hours, wherein each administration consists of five successive 1 ml sprays. For children, the formulation of the present invention can be preferably administered every four hours, wherein each administration consists of two or three 1 ml sprays. For infants, the formulation of the present invention can be administered every four hours, wherein each administration consists of one 1 ml spray.

According to still another specific embodiment, other administration routes for the formulation of the present invention include, without limitation, injection, topical application, intraocular application, nebulization or atomization, and the like.

According to further embodiment, the formulation of the present invention can be used during radiation or chemotherapy to protect the immune system. The mechanism of protection is not fully understood at the present. Radiation, chemotherapy, and surgery are the commonly used conventional cancer treatments. Both radiation and chemotherapy are highly damaging to fast growing cells in the body such as intestinal lining, bone marrow and cells of the immune system. After these treatments persons often have to be on very strong antibiotics in order to prevent infections. To avoid such damaging of the immune system, the use of the formulation of the present invention appears promising. The formulation can also be used in cases of surgical removal of certain tumours as an adjuvant therapy that results in a higher survival rate. Colds are viral diseases and formulation of the present invention can be used most commonly against viral conditions.

According to another further embodiment and within the spectrum of pharmaceutical formulation of the present invention, the activities of said composition has a unique enhanced activity for several viruses including, but not limited to HHV-6 variants (A and B), Epstein Barr Virus (EBV) and Cytomegalovirus (CMV). This enhanced activity provides for an improved immune response against active Human Herpesvirus-6 (HHV-6), Epstein Barr Virus (EBV) and Cytomegalovirus (CMV) infections. This leads to a decrease in viral activity and improved immune function (since HHV-6 and EBV are known to be immune suppressive viruses). Mammalian colostral fluids per se do not have this unique activity that is achieved after substantial isolation and purification of the peptides form it.

According to specific embodiment, the formulation of the present invention reduces symptoms such as, but not limited to Allergies, Alzheimer's, Benign Prostatic Hyperplasia, Cancer (adjunctive use only), Hypertension, Lupus (discoid and systemic), Thrush, Autism, Perthes Disease (active), Premenstrual Syndrome and Endometriosis, Prion Disease (Kuru and Creutzfeldt-Jakob Syndrome), Psoriasis, Sjogren's Syndrome, Spinal Muscular Atrophy, Thrombocytopenia (idiopathic and autoimmune), Topical applications (burns, infections, insect bites, diaper rash and herpetic lesions), Perthes Disease, Pharyngitis (Bacterial), Porphyria, Raynaud's Phenomenon, Sarcoidosis, Celiac Disease, Chronic Pancytopenia (inadequate levels of platelets), Crohn's Disease, Diabetes (type II), Fibro myalgia Rheumatica, Mononucleosis, Multiple Sclerosis, Perth's Disease, Rheumatoid Arthritis, Osteo Arthritis, Spinal Muscular Atrophy (Werdnig-Hoffman's Disease), Brown Recluse Spider Bite, Burns (1st, 2nd, 3rd Degree), Corneal Regeneration, Diarrhea, Endometriosis (inflammatory aspect), Guillain Barre' Syndrome, Hemolytic Anemia, Idiopathic Thrombocytopenia Purpura, Kuru (Prion Disease), Myasthenia Gravis, Lupus or Tuberculosis.

According to another specific embodiment, the formulation of the present invention cures diseases and conditions relating viral infection such as, but not limited to HIV, Hepatitis A&C, Rabies in Dogs, Acute Viral Infections, Chronic Viral Infections, Dengue Fever, Human Papilloma Virus, Parvo (canine), Pharyngitis (Viral), Rabies, SARS, Shingles, Viral Respiratory Infection, Plantar Warts, Colds and Flues, Dengue Fever, Lymphoma or Herpes Simplex I & II.

According to still another specific embodiment, the formulation of the present invention acts as and provides additional health benefits, such as, but not limited to, Anabolic, Anti-Aging, Anti-Fungal, Anti-Inflammatory, Anti-Microbial, Assimilation, Athletic performance, Body building, Bone density, Cytokines, Digestion, Elimination, Epithelial growth factor (EgF), Friendly flora, Insulin-like growth factor (IGF-1), Immunity, Immunoglobins, Interferon, Interleukin-10, Intestinal permeability, Lactoferrin, Methyl Sulfonyl Methane (MSM), Muscle tone, Prevention, Proline-rich Polypeptide (PRP), Recovery time, Retinoic acids, Skin elasticity, Soil-based organisms, Sports performance, Stamina, Terrain, Transfer factor, Transforming growth factors alpha & beta (TgF-A & TgF-B), Weight loss or Wound healing.

In a very specific embodiment, the formulation of the present invention is very useful in the case of cancer and cell-mediated immunity. Since cancer can be associated with a TH1-deficient state, use of pharmaceutical formulation of the present invention should be considered as a part of immune augmentation therapy in cancer. Factors that decrease cell-mediated immunity and TH2 dominance are age, cytotoxic cancer treatments, post-surgery stress, metastatic disease, etc. Cell-mediated immunity (CMI) can be a predictor of morbidity and mortality over the age of 60. In metastases or colon rectal carcinoma, CMI is predictive of survival. Decrease in cell-mediated immunity, along with an increase in circulating immune complexes, indicates unfavourable prognosis in cancer. In gynaecological cancer, chemotherapy had a decrease in immune perimeters (i.e., refractory decreased cell-mediated immunity), whereas immunotherapy using the formulation of the present invention maintains their immune perimeters at normal levels.

According to another embodiment, the formulation of the present invention shows improved cellular immunity in patients with immune defects. Since it augments TH1 or cell-mediated immunity, the PRP is helpful in these situations. For example, by conveying cell-mediated immunity against bladder and prostate tissue-specific antigen, the formulation of the present invention is efficacious in the treatment of stage D3 hormone-unresponsive metastatic prostate cancer.

According to still another embodiment, in the treatment of viral infections, the formulation of the present invention may provide a modality that works at a fundamental level. It induces interferon in patients with viral infection. Viral infections tend to have increased TH2 and decreased TH1. This is also seen with fungal infections, parasitic diseases, and cancer. Bacterial infections are associated with decreased TH2-dominant states. By stimulating TH1, peptides may be advantageous in the treatment of hepatitis. In hepatitis C, the activation of the TH2 dominance plays a role in the development of chronic hepatitis changes. TH1 stimulation may result in clearance of viral particles and improvement in the hepatitis. The measles can be treated successfully with non-specific peptides and symptoms may be ameliorated in some cases within 24 hours, without any side effects.

According to specific embodiment, the formulation of the present invention can be helpful for an impaired immune system that is subject to chronic infections, such as eczema, allergic diathesis, bronchitis, candidiasis or tonsillitis that eliminates need of frequent courses of antibiotics.

According to another embodiment, the formulation of the present invention may also be useful to ameliorate cases of recurrent, non-bacterial cystitis (NBRC) when treatment with antibiotics and nonsteroidal drugs is unsuccessful, and cell-mediated immunity to herpes simplex and Candida decreases. In Lyme disease, cytotoxic production of a TH2 phenotype is correlated to resistance, while that of a TH1 phenotype is correlated to susceptibility. This suggests that certain people have an immune glitch that makes their immune system prone to either the TH1 or TH2 pattern, and therefore more susceptible to different diseases. This may be precisely where formulation of the present invention, having immune-modulating effects, can be helpful.

According to specific embodiment, the formulation of the present invention can be used in chronic fatigue immune dysfunction syndrome, especially if a viral etiology is found. It may have varied success, although one may need to use increased dosages.

According to another embodiment, the treatment with the formulation, containing proline rich peptides, is dose dependent. In viral infections, may start with three times in a day. The dose is then tapered down to once in a day. That dose is maintained in cases of chronic viruses, chronic herpes infection, and chronic fatigue secondary to CMV or EBV, chronic colds, and impaired resistance. If there is any flare-up in viral infections, the dose can be increased to three times in a day. In allergic conditions, an adult may start with double dose thrice in a day, increasing to triple dose thrice in a day if symptoms get worse. Again, the dose may be tapered to a maintenance level with amelioration of allergic symptoms.

According to still another embodiment, in the cases of chronic fatigue syndrome may start on triple dose thrice in a day. One may need to increase the dose depending on the response. Doses of four to five times strength three times daily can be used as an adjunct cancer treatment for patients undergoing chemotherapy and/or radiation therapy, with a resulting decrease in cellular immune function.

Occasionally, a patient may experience flu-like symptoms, nausea, or gastrointestinal symptoms after taking the formulation of the present invention. Since the peptide of the formulation is a small and does not contain milk protein, allergic reactions are rare. These symptoms are classified as Jared Herxheimer mechanisms, and they probably signify a direct reaction of peptides of the formulation on gut or systemic pathogens. If patients are informed of these possible mild adverse reactions, they may more likely to continue treatment.

According to another aspect of the present invention also provided a method and a system for processing mammalian colostral fluids to isolate target peptides. The mammal can be a bovine, such as cow, goat, pig, buffalo, deer, or any other lactating mammal. The colostral fluids from lactating cows, but not limited, is preferred. Notably, the colostral fluids from other mammals can also be used to isolate the target PRPs.

The method includes the steps of passing mammalian colostral fluids through at least one ion exchange column or filter. The ion exchange column includes an anionic resin and/or a cationic resin. The ion exchange column or filter is selected to remove large particles and proteins, at a first pH level selected to remove such large particles and proteins at a maximum fluid pressure of 30 pounds per square inch (psi). In another arrangement, the maximum fluid pressure is less than 10 psi. The large particles and proteins are released from the ion exchange column or filter with a rinse solution having a pH selected for releasing the large particles.

The pH of the fluid then can be adjusted to be a value less than 5.0, and the fluid can be filtered with a microfilter having an initial pore size not greater than 300 nm or an ultrafilter having a pore size no greater than 20,000 Daltons. A pH of a retentate trapped by the filter can be adjusted to have a value between 6.5 and 7.0 and antibiotics can be washed from the retentate.

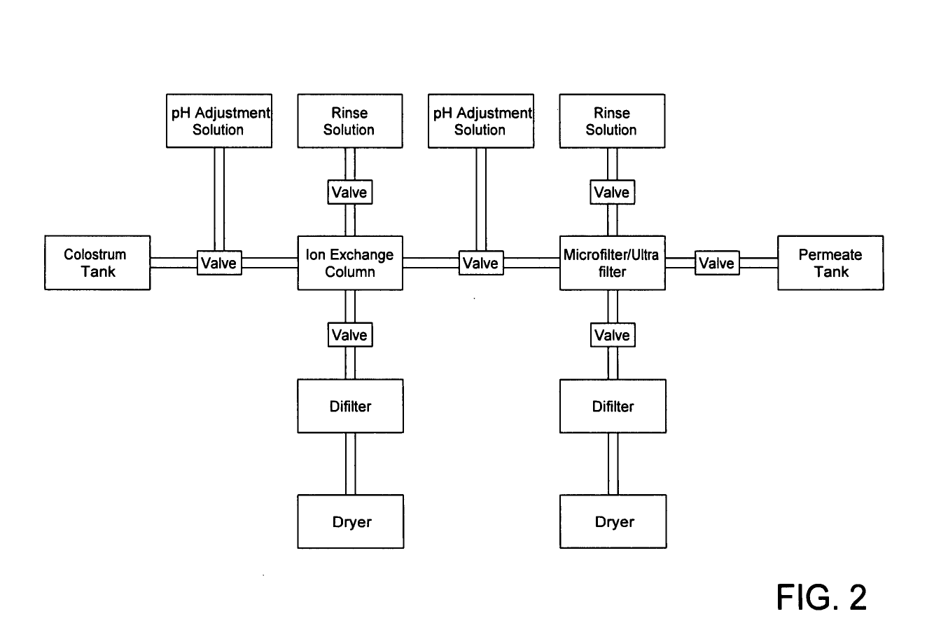

FIG. 2 is a block diagram of a processing system, which is useful for understanding the present invention.

For those references dealing with ways to isolate large molecules such as immunoglobulin, lactoferrin, etc., this is not surprising because such macromolecules are relatively hearty and capable of withstanding such pressures. Peptides, however, respond quite differently to high processing pressures. In particular, many types of peptides can be denatured at pressures ranging from about 210 kPa (approximately 30 psi) to about 690 kPa (approximately 100 psi). For example, peptides involved in the prevention of viral infections are among those that can be denatured at the lower end of this range of pressures (less than 10 psi). The term “Denatured”, with respect to a peptide, connotes an alteration or conformation change from the natural state due to, for example, physical forces, such as adhesion to another molecule(s), exposure to excessive temperature or pressure during processing, etc., chemical reaction, such as scission due to exposure to excessively acidic or basic conditions, enzymatic, degradation, and the like.

The present invention particularly relates to a method of isolating certain peptides and proteins from mammalian colostral fluids. In particular, the peptides and proteins isolated from the colostral fluids by filtering the it using one or more filters having a relatively small average pore size, for example an average pore size of 1.4 μm or less. In one embodiment, the peptides and proteins being isolated encounter a pressure that is equal to or less than 30 pounds per square inch (psi), and more preferably a pressure that is less than 10 psi. The peptides targeted for isolation, for example proline-rich-peptides and/or non-ionic peptides. Such peptides are important in various biochemical processes. The proteins targeted for isolation, for example, alpha-lactalbumin, beta-lactoglobulin, lactoferrin, lactoperoxidase, IgS, and immunoglobulins such as IgG, IgA, and IgM, etc.

Mammalian colostral fluids are potential sources for the target peptides and proteins. Advantageously, colostral fluids have relatively high levels of lactoferrin, immunoglobulins, and peptides, and colostral fluid is produced in relatively large quantities. Thus, for purposes of the following description of the isolation process, colostral fluid is used as a starting material.

The first colostral fluid withdrawn from a given mammal usually has a larger amount of the target proteins and peptides (per unit volume) than any subsequent colostral fluid collected from the same mammal. Thus, the first and second milking of a particular mammal provides a higher yield of the desired peptides in comparison to subsequent milkings. Although the production of the target peptides and proteins is not affected by the use of dairy cows which have had their utters specially treated with antibiotics or antigen-like materials that have more than a 30 day withholding period, the use of such dairy cows is not preferred because of potential contamination of the colostral fluid with antibiotics. Nonetheless, if such dairy cows are used, the antibiotics or materials are removed from the colostral fluid using filtration technologies.

The colostral fluid is pre-processed. The colostral fluid is collected and stored under suitable storage conditions. Specifically, the colostral fluid preferably is collected under conditions that prevent gross contamination by bacteria. The collection and storage conditions are those that are appropriate for the storage of milk for human consumption. The conditions are consistent with applicable governmental norms, for example, FDA guidelines.

Where pumps are employed in a given step, they preferably are of a type that does not have impellers or other features capable of producing a shearing effect. In the design of a specific process, each aspect preferably is considered and tailored to prevent damage to the target peptides and proteins. Once the raw colostral fluid farm tank has been emptied the tank is cleaned with an automatic CIP system through a spray ball. The use of a tank that does not have sharp corners is used to facilitate cleaning of the tank and minimize build up of colostral fluid in the corners of the tank.

During processing, it is preferred that the fluid pressures which are experienced by the target peptides at each part of the process stay equal to, or below, 30 psi. Keeping the fluid pressures experienced by the target peptides at or below about 10 psi can minimize denaturing of the target peptides. To provide these relatively low operating pressures while maintaining industrially acceptable processing speeds, running the process in the form of a batch (as opposed to continuously) is preferable. Where batch processing is used, typical amounts ranges from about 375 to about 37,500 L (100 to 10,000 gallons), depending on down-line processing speed.

There is a further benefit by immediately placing the raw material under refrigeration. Cooling the raw material to less than 45° F. in less than 4 hours reduces bacterial growth in colostral fluid. Inline plate heat exchangers or chilled water systems can also be used to immediately cool the colostral fluid to a desired temperature. However, the invention is not so limited and other cooling methods can also be used to economize the process of isolation.

Freezing of colostral fluid reduces the efficacy or concentration of the natural peptides and is therefore not desired because the freezing of the water within the raw material damages many of the peptides and globular proteins. In particular, water crystals formed when the colostral fluid is frozen cuts through the proteins, and thus increase the degree of denaturing. This reduces the yield of target peptides and proteins. Nevertheless, the use of previously frozen colostral fluid is not optimal; still it can be used and is therefore within the scope of the present invention.

Processing of the colostral fluid is performed proximate to or remote from the point of collection. In the case that processing is performed remote to the point of collection, transportation of the raw material preferably occurs under conditions suitable for handling of the raw material without significant denaturing of the target peptides and proteins and without excessive bacterial growth. The raw material can be transported using methods known to those skilled in the art.

Testing can be performed on the colostral fluid to measure the concentration of target proteins and/or target peptides within the raw material. For example, when attempting to collect colostral fluid from a mammal, an amount of milk may also be collected. Since milk has a lower concentration of the target peptides and proteins than colostral fluid, greater milk content within the raw material lowers the yield of the target peptides and proteins. Specific gravity testing is used to measure the amount of milk present in the raw material, and thus provide an indication of the quality of the raw material for the intended purpose. Other methods used to indicate the quality of the raw material are high-pressure-liquid-chromatography, radial immunodiffusion (RID) assay, and/or enzyme-linked immunosorbent assay (ELISA) to accurately determine the levels of immunoglobulins present. The testing of the raw material is implemented as part of a quality control program.

The amount of colostral fluid collected from a cow is limited to no more than approximately 15 L (4 gallons), collected within the first 24 hours after birth. In the case that the colostral fluid is transported to a processing facility remotely located from the location of the colostral fluid collection, it can be advantageous to maintain the colostral fluid below about 45 degrees Fahrenheit (7 degrees Centigrade) at all times to prevent excessive bacteria growth, but it is preferred that freezing of the colostral fluid be avoided to prevent denaturing of the proteins and peptides. Further, it also can be advantageous to process the colostral fluid soon after collection to insure a high quality product.

It is preferred that the colostral fluid should arrive at the processing facility within 72 hours of collection. The colostral fluid can be pasteurized using an automated legal high temperature short residence time (HTST) pasteurizing system, or ultra-high temperature (UHT) pasteurizing system, but preferably not batch pasteurized. The raw material is stored in a suitable storage container for processing. The storage container is a tank made of inert stainless steel material.

The process also isolates colostral whey or colostral sera from the colostral fluid for further processing. Colostral whey is derived from separation technologies from any pre-curing of the colostral fluid. Colostral sera are derived from separation technologies from any non-curing processes of the colostral fluid. When colostral fluid is collected from more than one mammal, the colostral fluid is stirred with some type of stirring mechanism, for example a paddle or stirrer blades, or a pumping motion, to gently mix the raw material. Where stirrer blades are used as the stirring mechanism in the preceding step, slow rotation of the blades and a backward angling of the blades provides a gentle mixing to minimize damage to peptides and proteins. Where pumping is used as the stirring mechanism in the preceding step, slow rotation of the impellers with low shear impeller designs provides a gentle mixing to minimize damage to peptides and proteins.

Such mixing can help to counter inconsistencies between colostral fluid from the different mammals and to provide a more uniform temperature because a particular cow might be deficient in one or more particular peptides, a blending of colostral fluid from many cows provides a given colostral blend that contains all of the target peptides and proteins. Further, both cow and heifer colostral fluid is mixed together. Colostral fluid containing blood of non-genetically modified mammals typically does not have a high concentration of the target peptides, however.

After blending, the colostral fluid blend is moved to a first phase of a reduction process, which involves separating and removing much fat from the colostral fluid. Various means, such as separators, centrifuges, chemical, hydrophobic, supercritical CO₂ or liquid-liquid extractions are available to accomplish this task.

The defatted blend is conveyed to a stainless steel tank as a curdling vessel where it is gently stirred. During this process the temperature of the defatted blend can be raised to between about 90° F. (32° C.) and about 99° F. (37° C.) in preparation for curdling. Curdling involves the coagulation of the majority of casein remaining in the blend. It generally is accomplished by addition of rennin, an enzyme-rich extract from the stomachs of calves, or an acid such as HCl to the warmed colostral fluid blend. Once the rennin or acid is added, a curd gradually begins to form soon after stirring of the blend is stopped. As the curd forms, it rises to the surface, producing a soft white cake or crust sitting or floating on whey. The whey, which is the desired product from this step, is drained away from the curdling vessel. Alternatively, a defatted colostral fluid stream is micro-filtered and/or ultra-filtered instead of curding to extract the same peptides.

The remaining curd can be cut into small pieces by activating a stirring mechanism in the tank. The broken curd is conveyed away from the tank through pipes made of an inert stainless steel material, to a large screen, which also is made from a relatively inert material. Any whey trapped in the curd passes through the screen and, optionally, is added into the whey collected previously.

The collected whey or sera is passed through a fines reducer or a clarifier to exclude more of the small pieces of curd, which is conveyed away from the curdling vessel during removal of the whey. This additional step, although certainly not required, is beneficial because it increases the service interval for the filter media described below. The whey is conveyed to the next step of the reduction process through stainless steel pipes.

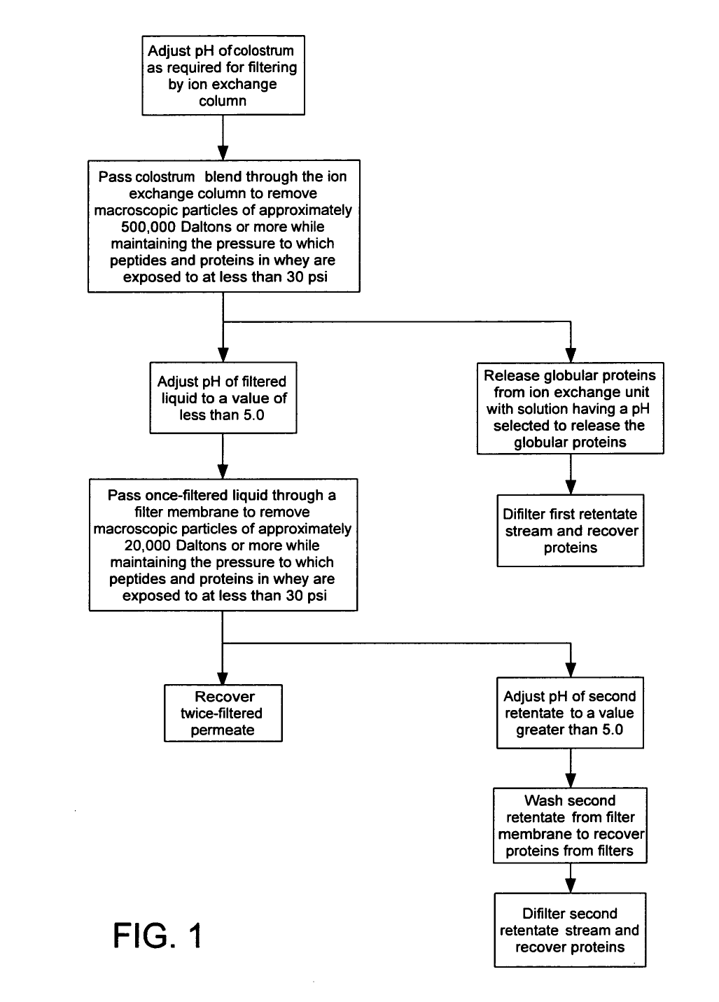

Making reference to the processing system 200 of FIG. 1, the pre-processed colostral whey or sera 202 undergoes further processing using ion exchange chromatography to isolate desired proteins from the colostral whey 202. The pH of the colostral whey 202 is adjusted as required for large protein sorption by one or more ion exchange columns 204, which is a cationic ion exchange column or an anionic ion exchange column. The ion exchange column 204 comprises resin, which is in the form of a bead of uniform size or with a wide standard deviation of sizes, or a non-spherical shape of similar or varying sizes. The cationic resin is an SP or SM type resin, and an anionic resin is a QEA or Q type resin. The resin is packed with adequate bulk density or contained within a containment vessel so as to allow adequate flow of the colostral whey 202.

A pH adjustment solution 206 is added to the colostral whey 202 until a suitable pH of the colostral whey 202 is reached. Notably, the pH of the colostral whey 202 can affect the surface chemistry of proteins contained within the colostral whey 202, and give the surface of the proteins a net negative charge, a net positive charge, or a net neutral charge. A suitable acid for lowering the pH of the colostral whey 202 is food grade citric acid, phosphoric acid, or lactic acid. Alternatively, a suitable base can be added to the whey to raise the pH. The suitable bases are potassium hydroxide, calcium hydroxide and sodium hydroxide. Notably, the use of potassium hydroxide adds potassium to the colostral fluid, which protects proteins during a subsequent pasteurization process, if such a process is used. A suitable valve 214 and/or suitable mixing pumps can be used to mix the pH adjustment solution 206 with the colostral whey 202.

The colostral whey 202 is passed through the ion exchange column 204 at a pressure less than or equal to 30 psi, and more preferably below 10 psi, to isolate one or more globular proteins between 10,000 and 500,000 Daltons. The proteins include alpha-lactalbumin, beta-lactoglobulin, lactoferrin, immunoglobulins and other whey proteins. A cationic ion exchange column comprises a positive resin. In consequence, lactoferrin and lactoperoxidase are sorbed when the pH of the whey solution is between about 6.5-7.0. Moreover, all whey proteins can be sorbed within the cationic ionic exchange column when the pH is less than 4.0. The pH is in the range of about 3.0 to 4.0. An anionic ion exchange column comprises a negative resin. In this arrangement, all whey proteins are sorbed when the pH of the whey solution is between about 6.5-7.0. Typically, little or no proteins are sorbed in an anionic exchange column when the pH of the solution is less than 4.0.

Proteins sorbed in the resin are globular proteins that typically do not bind to the resin. Since the proteins do not bind to the resin, the proteins are recovered from the ion exchange unit 204 by passing a rinse solution 216 having a pH selected to release the proteins. Particularly, the rinse solution 216 changes the charge of the resin in the ion exchange column 204 and causes the resin to release the proteins into a retentate stream. Solutions are also used to change the net charge of the surface of the proteins so that the proteins repel the cationic or anionic resins. The retentate stream with the liberated proteins is then difiltered to remove any salts that may remain in the solution. The retentate stream is passed through one or more filters 218 which have pore sizes greater than the globular proteins in the retentate stream. The retentate stream then is dried to recover the globular proteins. The proteins are concentrated to between 15-30% total solids. The proteins are then passed through a dryer 220 and subjected to freeze or low-heat indirect steam spray drying. An evaporator is used to concentrate further the retentate stream. Suitable valves 214 and/or suitable fluid pumps can be used to control fluid flow within the system 200.

The whey or sera need not be subjected to fluid pressures in excess of normal bodily fluid pressures in this process, thus preserving the native states of the proteins and peptides contained in the whey. The resulting filtrate or flow-through stream is therefore more effective in cellular interactions in comparison to a filtrate filtered at pressures, which are significantly higher than the pressures the bodily fluids experience within the mammal from which they are extracted. Also, the yield of non-ionic peptides from the whey or sera using ion exchange technologies, as will be described below, can be between 80-85% when compared to the original number in the raw colostral fluid liquid. When membrane filtration techniques are exclusively used to isolate the proteins, a peptide yield of about 45-50% can be expected.

To achieve adequate separation while maintaining operating pressures at or below about 30 psi, for example below about 10 psi, relatively short filtration units is used to minimize back pressure. The filtration units is on the order of about 0.3 m to 1.5 m in length ion exchange columns with fixed beds, packed beds or expanded bed designs, having bed depths not exceeding 20 cm to 25 cm, thus minimizing the pressure drop across the bed, preferably less than 5 psi. Notably, dynamic or moving ion exchange beds do not have such pressure drops. However, the yield is less with dynamic beds as compared to static or fixed beds. Specifically, there is a higher concentration of proteins in the whey using dynamic beds as compared to exposing the stream of whey to fresh resin in lower sections of a fixed bed.

In one arrangement, two or more ion exchange columns are cascaded in series. The use of cascaded ion exchange columns provides a longer contact time for the flow-through protein solution. Moreover, multiple ion exchange columns are connected in parallel to increase throughput and scalability of a commercial ion exchange process. Ion separation technologies as described herein are used before or after the filtration steps as needed to purify individual or multiple protein streams, depending on the order in which it is desired to remove the proteins. Notably, the order of ion exchange and filtration does not affect production of desired peptides.

Optionally, the diameter of the fixed ion exchange beds can be minimized to less than 30 cm, to prevent uneven bed depths of resin across the resin bed to minimize “uneven lateral resin migration”. Minimizing the lateral resin migration helps to maintain a uniform pressure drop through the ion exchange bed.

In lieu of ion exchange columns 204, tangential flow or cross-flow filters are used; tubular microfilter unit also can be used. Tangential or cross flow filtration is generally preferred over dead-end filtration because the latter can result in unacceptable pressures unless throughput speeds and volumes are kept quite low. Additionally, tangential flow units do not result in all particles being trapped in the filter membrane, i.e., certain large particles merely pass along the exterior of the membrane and never get retained in a pore; this extends the operation period for a given filter unit.

Micro filters as a class generally are used to remove substances that range in size from about 0.1 to about 1.4 μm. By selecting a filter with an average pore size of from 0.1 to 0.45 μm, one can achieve the desired result of removing most materials having a molecular weight of approximately 500,000 Daltons or more, which includes almost all bacteria but very few proteins. In fact, proteins the size of antibodies and smaller pass through this size of filter pore with the greater portion of the water present in the whey.

Microfilter membranes can be made from a wide variety of materials and are commercially available from numerous sources. Membranes can have inner permeate tube diameters of about 0.05 inch to 2.0 inches and outer diameters of about 1.0 inch to 12.0 inches. Acceptable micro filters for such units include ceramic filters, metallic, Teflon, polyethylsulphone (PES) 9 other polymeric spiral wound, tubular, polyplastic membranes, etc).

The colostral whey or sera 202 is conveyed, again preferably by inert stainless steel pipes, to the next step in the reduction process. Here, suspended materials that range in size down to that which can be seen only with a relatively powerful microscope can be removed, for example those particles greater in size than 100,000 Daltons. The pH of colostral whey 202 again is adjusted with a pH adjustment solution 208. A suitable base or suitable acid is added to the colostral whey 202 until the pH is in the range from about 4.5 to about 5.0. Optionally, it is preferable that the pH be in the range from about 4.55 to about 4.70, and most preferably of about 4.6. This slightly acidic pH has been found to be sufficient to kill or disable most bacterium that might have made its way through the processing or that might have been in the container used to hold the filtrate product. The need for preservatives and anti-bacterial, yeast and mold inhibitors is therefore reduced or eliminated.

The twice pH adjusted colostral whey 202 is passed through microfilter(s) and/or ultrafilter(s) 210 to remove macroscopic particles of approximately 20,000 Daltons or more while maintaining the pressure to which the remaining proteins and peptides in the colostral whey are exposed to at less than 30 psi. An exemplary separation process which implements a tangential flow unit having an ultrafilter membrane. Nanofiltration or reverse osmosis can also be used. However, nanofiltration and reverse osmosis are expensive and typically require higher operating pressures than ultrafiltration. The tangential flow filtration results in lower operating pressure than dead-end filtration in both the cases.

A tangential flow ultrafilter unit is similar in design to the previously described microfilter unit, except that its filter can have smaller pores. The average pore size of this type of filter ranges from about 1,000 to about 100,000 Daltons. An ultrafilter for such a unit includes, for example, a spiral wound filter. The membranes of the filter are made of cellulose materials, fluoro polymers, polysulfones, or any other suitable material.

In some instances, the acidic pH of the liquid will cause the pore sizes of polymer or non-rigid plastic filter membranes to shrink. A pH of 4.6 causes the pores of certain microfilter membranes having an initial pore size greater than 120,000 Daltons to shrink to 50,000 Daltons, or smaller. The average pore size is in the range from 300 nm to 450 nm (0.3 μm to 0.45 μm). A preferred average pore size after shrinkage is from 15,000 to about 30,000 Daltons, with 20,000 Daltons being a highly preferred average pore size. Use of such relatively small pores with acidic pHs, as described, results in the removal of most proteins (including antibodies) and endotoxins, which might have been included in the original colostral blend. More particularly, transmission of the antibiotic residues through the membrane is inhibited, and the residues thus remain in the retentate. In consequence, the acidic permeate that is produced is free from antibiotic residues and thus is as a good source of proline-rich-polypeptides.

In the two preceding filtrations, the temperature of the ion exchange columns and filtration units is maintained as high as about 52° C. to control bacterial growth. Alternatively, process temperatures for all unit operations is maintained to be not higher than 0.50 to 5° C. Use of such relatively low temperatures has been found to keep any fat remaining in the whey in macroscopic globules that do not even enter, and thus occlude, the pores of the filters. The low temperature also maintains low growth of microbes in the process lines and fluids. The antibiotic free permeates is then collected as products and set aside from further Ion exchange separations.

Where the ultrafiltration step employs a filter having an average pore size of no more than about 20,000 Daltons, the filtrate product is essentially free of proteins, peptides, and biochemical macromolecules that have a molecular weight of 20,000 Daltons or greater. Penicillin G like antibiotics will be removed from the filtrate product during the ultrafiltration step. More preferably, the filtrate product is essentially free of peptides or proteins having molecular weights of about 10,000 Daltons or greater, and most preferably at least 90% of the component peptides of the filtrate product have molecular weights not more than about 2,500 Daltons. This is important for several reasons, one of which is that the filtrate then is substantially free of molecules that can act as points of agglomeration for the desired peptides. A peptide that has agglomerated to other peptides or to larger proteins usually is denatured. Similarly, peptides subject to bacterial-induced enzymatic degradation also can be denatured and, accordingly, the filtrate product preferably is essentially free of such bacteria. Moreover, the filtrate product preferably has not more than about 10% of its component peptides in a denatured state. Even lower concentrations of denatured peptides are desirable.

The antibiotic free low pH filtered permeate obtained is collected in a permeate tank 212 until the flow rate drops significantly and used for further processing as liquid or powder colostral fluid products. The resulting antibiotic free low pH permeate (filtrate product) which passes through the micro filters and/or ultra filters is collected until the flow rate drops significantly and used for further processing as liquid or powder colostral fluid products.

Optionally, the filtrate product can be further preserved by adding food grade 0.1% EDTA to inhibit bacterial growth and 0.1% Potassium Sorbate to minimize any yeast and mold from growing in the final product, or hydrogen peroxide, or some other suitable preservative. If desired, the filtrate product may have the flavor profile changed by adding a flavor ingredient such as vanilla, chocolate, strawberry, pina-colada, etc. This may make the filtrate product more palatable as an ingested liquid, spray, capsule, or lozenge. If the filtrate is to be used as topical liquid or topical spray only, scent could be added to the filtrate. As a final treatment the filtrate product can be further filtered one or more times using a filter not more than 0.45 μm to catch any potential remaining microbes. The filtrate then can be aseptically packaged. At all stages of the processes Good Manufacturing Practices (GMPs) should be followed to preserve the integrity of the final filtrate product.

Preferably, each milliliter of filtrate product contains at least 0.3 g of total protein, more preferably at least 0.35 g protein and most preferably from 0.36 to 0.38 g of peptides. Although this amount seems small, it has been found to provide a full array of target peptides, thus providing maximum benefit to the human or animal which receives a dose thereof.

In the more preferred embodiment, the retentate next is collected from the micro filters/ultrafilters. The pH of the retentate collected in the micro filters and/or ultra filters is adjusted to be above 5.0, and preferably in the range 6.5 to 7.0. The pH is adjusted using a basic solution, such as food grade calcium hydroxide, potassium hydroxide and/or sodium ammonium hydroxide. The retentate is released from the microfilter(s) and/or ultrafilter(s) 210 with a rinse solution 222 to recover filtrate product from the filters 210. The rinse solution 222 is a salt solution or other suitable permeate stream with some ionic strength to wash antibiotics from the retentate. The retentate stream with the liberated filtrate product is then difiltered to remove any salts that may remain in the solution. For example, the retentate stream can be passed through one or more filters 224 which have pore sizes greater than the filtrate product in the retentate stream.

Washing the retentate with an ionic solution at neutral pH and three times the volume of the retentate is sufficient to reduce the antibiotic levels to less than 5 ppb for all types of penicillins, which is below the United States Department of Agriculture (USDA) maximum level for acceptable milks. Moreover, the rinse solution 222 circulated to repeat the washing cycle could help to further reduce the antibiotic levels. In one arrangement, the rinse solution 222 can be a waste stream from another dairy process. The wash/difilter process is repeated until the antibiotics reach a low enough level. The rinse stream that passes through the microfilters and/or ultra filters is collected until the flow rate drops significantly, and discarded as a waste product.

The retentate is then dried to recover the filtrate product. The filtrate product is passed through a dryer 226 and subjected to freeze or low-heat indirect steam spray drying. An evaporator is used to concentrate further the filtrate product. Again, suitable valves 214 and/or suitable fluid pumps (not shown) can be used to control the fluid flow. All stages in the process can adhere to a Hazard-Analysis and Critical-Control Program (HACCP) and Good Manufacturing Processes (GMPs) consistent with the recommended procedures of the USDA and Food and Drug Administration (FDA).

The mammal that produces the raw material used for processing is a mammal, which has immunity to an antigen. The mammal can be rendered immune by injecting an antigen or a protein specific for the antigen into the mammal. In another arrangement, the mammal can be exposed to the antigen before the mammal commences lactation. The antigen can be a virus or can be derived therefrom. For example, the virus can be the Human Immunodeficiency Virus (HIV, type 1 or type 2). The virus also can be a planar warts virus, an influenza virus, a cold virus, or any other virus.

Exposure by the mammal producing the raw material to the virus can result in peptides and proteins being produced which can be used to fight ailments. Accordingly, the formulation of the present invention can be orally administered to maintain wellness or to induce recovery from a wide range of infectious and progressive disease processes. For example, but not limited to, the formulation of the present invention can be used to treat Allergies, Arthritis, Benign Prostatic Hyperplasia (such as the inflammatory aspect), Cancer (for example for adjunctive use), Celiac Sprue, Crohn's Disease, Diabetes Type II, Hypertension, Lupus (Discoid and Systemic), Multiple Sclerosis, Perthes Disease (Active), Premenstrual Syndrome and Endometriosis, Prion Disease (Kuru and Creutzfeldt-Jakob Syndrome), Psoriasis, Sjogren's Syndrome, Spinal Muscular Atrophy, Thrombocytopenia (Idiopathic and Autoimmune), Topical Applications (burns, wounds, infections, insect bites, diaper rash and Herpetic Lesions), Acute Viral Infections, and numerous other ailments. Further, peptides and proteins isolated from the raw material can be used to treat digestive problems. Still, there are a number of other medical uses for such proteins, and the invention is not so limited.

The above list of conditions and diseases are simply a sample of what can be treated indirectly by a course of formulation treatment, and is by no means limiting. Moreover, in recent years, peptide treatment has been found to be beneficial for a wide variety of ailments. In the peptide treatment of non-viral diseases or conditions, for example, it is believed that the peptides can be used to provide correct information to cells for mammal cellular function, and thus better prepare the cells for the treatment of the conditions and diseases. In viral treatments, the peptides can act as protectors to the cellular surface by preventing the viral ligand proteins from attaching to a receptor on a cellular surface.

The following examples represent potential uses for peptides isolated using the above process for treating a number of different medical conditions. It should be noted that the isolated peptides can be used for numerous other treatments and the examples listed are by no means exhaustive.

### EXAMPLES

### Example 1

Allergies Typical dose: three sprays (5 ml). Typical administrations per day: 2 typical interval of initial response: 1-3 days typical interval to benefit plateau: 3-7 days typical response: 2-3 days No reduction or elimination of other therapeutics until justified by condition of patient. Example 2: Arthritis Typical dose: three sprays (5 ml). Typical administrations per day: 2 typical interval of initial response: 7-21 days typical interval to benefit plateau: 42-56 days typical response: 2-3 days No reduction or elimination of other therapeutics until justified by condition of patient. Example 3: Benign Prostatic Hyperplasia (inflammatory Aspect) Typical dose: three sprays (5 ml). Typical administrations per day: 2 typical interval of initial response: 7-14 days typical interval to benefit plateau: 14-28 days typical response: 2-3 days No reduction or elimination of other therapeutics until justified by condition of patient. Example 4: Cancer (Adjunctive use only) Typical dose: three sprays (5 ml). Typical administrations per day: 2 typical interval of initial response: 7-14 days typical interval to benefit plateau: N/A Typical response: 2-3 days No reduction or elimination of other therapeutics until justified by condition of patient. Example 5: Celiac Sprue Typical dose: three sprays (5 ml). Typical administrations per day: 2 typical interval of initial response: 1-3 days typical interval to benefit plateau: 3-7 days typical response: 2-3 days No reduction or elimination of other therapeutics until justified by condition of patient. Example 6: Crohn's Disease Typical dose: three sprays (5 ml). Typical administrations per day: 2 typical interval of initial response: 7-14 days typical interval to benefit plateau: 42-56 days typical response: 2-4 days No reduction or elimination of other therapeutics until justified by condition of patient. Example 7: Diabetes Type II Typical dose: three sprays (5 ml). Typical administrations per day: 2 typical interval of initial response: 1-7 days typical interval to benefit plateau: 14-28 days typical response: 2-3 days No reduction or elimination of other therapeutics until justified by condition of patient. Example 8: Hypertension Typical dose: three sprays (5 ml). Typical administrations per day: 2 typical interval of initial response: 7 days typical interval to benefit plateau: 28-56 days typical response: 2-3 days No reduction or elimination of other therapeutics until justified by condition of patient. Example 9: Lupus (Discoid and Systemic) Typical dose: three sprays (5 ml). Typical administrations per day: 2 typical interval of initial response: 7-14 days typical interval to benefit plateau: 28-58 days typical response: 2-3 days No reduction or elimination of other therapeutics until justified by condition of patient. Example 10: Multiple Sclerosis Typical dose: three sprays (5 ml). Typical administrations per day: 2 typical interval of initial response: 7-28 days typical interval to benefit plateau: 28-180 days typical response: 2-3 days No reduction or elimination of other therapeutics until justified by condition of patient. Example 11: Perthes Disease(Active) Typical dose: three sprays (5 ml). Typical administrations per day: 2 typical interval of initial response: 1-2 days typical interval to benefit plateau: 7-28 days typical response: 3-4 days No reduction or elimination of other therapeutics until justified by condition of patient. Example 12: Premenstrual Syndrome and Endometriosis Typical dose: three sprays (5 ml). Typical administrations per day: 2 typical interval of initial response: 1-2 days typical interval to benefit plateau: 14 days typical response: 2-4 days No reduction or elimination of other therapeutics until justified by condition of patient. Example 13: Prion Disease (Kuru and Creutzfeldt-Jakob Syndrome) Typical dose: three sprays (5 ml). Typical administrations per day: 2 typical interval of initial response: 12-28 days typical interval to benefit plateau: 28-56 days typical response: 2-3 days No reduction or elimination of other therapeutics until justified by condition of patient. Example 14: Psoriasis Typical dose: three sprays (5 ml). Typical administrations per day: 2 typical interval of initial response: 3-7 days typical interval to benefit plateau: 28-56 days typical response: 2-3 days No reduction or elimination of other therapeutics until justified by condition of patient. Example 15: Sjogren's syndrome Typical dose: three sprays (5 ml). Typical administrations per day: 2 typical interval of initial response: 7-14 days typical interval to benefit plateau: 14-28 days typical response: 3-4 days No reduction or elimination of other therapeutics until justified by condition of patient. Example 16: Spinal Muscular Atrophy Typical dose: three sprays (5 ml). Typical administrations per day: 2 Typical interval of initial response: 7-14 days Typical interval to benefit plateau: 180 days Typical response: 2-3 days Increased consumption of water should be encouraged in adult patients with this disease Example 17: Thrombocytopenia (Idiopathic and Autoimmune) Typical dose: three sprays (5 ml). Typical administrations per day: 2 typical interval of initial response: 1-2 days typical interval to benefit plateau: 4-10 days typical response: 3-4 days No reduction or elimination of other therapeutics until justified by condition of patient. Example 18: Topical Applications (burns, wounds, infections, insect bites, diaper rash and Herpetic Lesions) Typical method of administration spray or apply with a sterile pad to affected area. Typical administrations per day: three sprays (3-5 ml) Typical interval to initial response: 4-24 hours Typical interval to benefit plateau: Variable with condition Typical response: 3-4 days typical observations include accelerated healing and reduced tendency to scar. Example 19: Acute Viral Infections Typical dose: three sprays (2 ml). Typical administrations per day: 6 (every four hours) Typical interval of initial response: 4-24 hours typical interval to benefit plateau: 2-7 days typical response: 3-4 days No reduction or elimination of other therapeutics until justified by condition of patient. Example 20: Chronic Viral Infections (including HIV and SARS) Typical dose: three sprays (2 ml). Typical administrations per day: 6 (every four hours) Typical interval of initial response: 4-24 hours typical interval to benefit plateau: 2-7 days typical response: 3-4 days No reduction or elimination of other therapeutics until justified by condition of patient.

### Example 2

Combined results of clinical trials as function of oral administration of the formulation containing PRP in an effective amount as substantially herein before described on HIV patients are summarized in following tabular data These results are for thirty-nine (39) patients those showed symptoms initially and for which later data was obtained. The Phase I trial was conducted at the Infectious Disease Clinic in Dayton, Ohio in February to April 1996. The Phase II and III trials were conducted at the University of Nairobi in Nairobi, Kenya. From March to August 2000. The results as a function the formulation of the present invention containing PRP on clinical symptoms, physical findings, viral load and CD-4 count are depicted in Tables I, II, III and IV, respectively. The results confirmed the effectively of the formulation in reduction of the clinical symptoms score, physical findings score and viral load, and improvement in CD-4 count.

TABLE I
Clinical Symptoms Score

| | Initial | 7 days | 14 days | 21 days | 28 days | 43 days | 60 days | 75 days | 90 days |
|---|---|---|---|---|---|---|---|---|---|
| Total Patient | 35 | 34 | 33 | 32 | 31 | 26 | 20 | 18 | 17 |
| Reports | | | | | | | | | |
| Score | 6.1 | 4.0 | 3.2 | 2.7 | 1.8 | 1.5 | 1.2 | 1.3 | 1.3 |
| Percent | | 34 | 48 | 55 | 69 | 75 | 80 | 79 | 79 |
| Reduction | | | | | | | | | |
| Percent | | 35 | 50 | 55 | 60 | 65 | 70 | 80 | 80 |
| reduction | | | | | | | | | |
| after Phase | | | | | | | | | |
| III trial | | | | | | | | | |

TABLE II
Physical Findings Score

| | Initial | 7 days | 14 days | 21 days | 28 days | 43 days | 60 days | 75 days | 90 days |
|---|---|---|---|---|---|---|---|---|---|
| Total Patient | 30 | 29 | 28 | 28 | 27 | 19 | 13 | 13 | 13 |
| Reports | | | | | | | | | |
| Score | 4.0 | 3.7 | 3.3 | 2.9 | 2.5 | 2.3 | 2.1 | 1.9 | 1.6 |
| Percent | | 7.5 | 18 | 28 | 38 | 43 | 49 | 53 | 60 |
| Reduction | | | | | | | | | |
| Percent | | 7.5 | 20 | 30 | 40 | 45 | 50 | 55 | 60 |
| reduction | | | | | | | | | |
| after Phase | | | | | | | | | |
| III trial | | | | | | | | | |

TABLE III
Viral Load (viral load count is available on only seven patients from the Phase II trial)

| | Initial | | | | | | | | |
|---|---|---|---|---|---|---|---|---|---|
| | viral load | 7 days | 14 days | 21 days | 28 days | 43 days | 60 days | 75 days | 90 days |
| Patient | 92,448 | 71774 | 51110 | 30428 | 9,755 | | 445* | | 13 |
| Patient | 28,049 | 21193 | 14337 | 7481 | 625 | | | | 1.6 |
| Patient | 33,093 | 25058 | 16785 | 8561 | 239* | | | | |
| Patient | 470* | | | | 37,913 | | | | |
| Patient | 439* | | | | | | 175 | | |
| Patient | 59,821 | | | | | | | | 320* |
| Patient | 40,381 | | | | 180* | | | | |
| Percent | | 12.5 | 17.5 | 27.5 | 35 | 45 | 55 | 62.5 | 70 |
| reduction | | | | | | | | | |
| Phase III | | | | | | | | | |
| trial | | | | | | | | | |

TABLE IV
CD4 Count

| | Initial | | | | | | | | |
|---|---|---|---|---|---|---|---|---|---|
| | CD4 | 7 days | 14 days | 21 days | 28 days | 43 days | 60 days | 75 days | 90 days |
| Patient | 74 | | | | 153 | | 121 | | |
| Patient | 274 | | | | 282 | | | | |
| Patient | 245 | | | | 301 | | | | |
| Patient | 60 | | | | 47 | | | | |
| Patient | 101 | | | | | | 117 | | |
| Patient | 211 | | | | | | 291 | | |
| Percent | | 10 | 25 | 35 | 50 | 52.5 | 57.5 | 62.5 | 75 |
| Improvement | | | | | | | | | |
| after Phase III | | | | | | | | | |

Measuring the CD4 counts in patients with HIV is a valid marker to indicate the progression of the infection. However, the CD4 test is only one of several indicators of wellness. In the Phase III trial the CD4 levels may not normalize sooner but rather than later.

The CD4 test is performed on a small sample of blood taken from a vein in the arm, then calculated the number of CD4 cells per cubic millimetre of blood. The test measures the number of CD4 cells circulating in the blood, which is approximately only 2% of their total number in the body. The majority of CD4 cells are to be found in the lymph nodes. Note, PRP supplementation may decrease the CD4 count in the blood stream initially because the virus is prevented from entering the T-cells particularly in the lymph nodes and more viruses then appear in the blood stream, thus there is a radical increase in viral load in the blood stream and consequently a potential drop in CD4 counts as well.

While the preferred embodiments of the invention have been illustrated and described, it will be clear that the invention is not so limited. Numerous modifications, changes, variations, substitutions and equivalents will occur to those skilled in the art without departing from the spirit and scope of the present invention as described in the claims.

I claim:

1. An immunologically active peptide fragments of a proline-rich polypeptide isolated from mammalian colostral fluid for immuno-regulatory properties characterized by PRP1, PRP2, PRP3, and to a lesser level PRP4 and PRP5, comprising short chain proteins with a high concentration of the amino acid Proline that support the regulation of the thymus responsible for the normal development of immunologic function in the body.

2. The immunologically active peptide fragments of claim 1, which contain tiny protein molecules produced by immune T-cells, thereby playing a central part in promoting immune system strength and effectiveness.

3. The immunologically active peptide fragments of claim 1, which contains a set of messaging molecules, thereby conveying immune information within an individual's immune system.

4. The immunologically active peptide fragments of claim 1, which contains signal proteins, thereby transferring immunity from one person to another that is vital to the sound health.

5. The immunologically active peptide fragments of claim 1, which contain small immune messenger molecules produced by mammals.

6. The immunologically active peptide fragments of claim 1, which contain small peptides of about at least eight amino acid residues.

7. The immunologically active peptide fragments of claim 1, which directly support the Natural Killer (NK) cells of the immune system.

8. The immunologically active peptide fragments of claim 1, which provides NK cells specially equipped to locate and kill disease cells, thereby providing front line of defense.

9. The immunologically active peptide fragments of claim 1, wherein peptides are not species-specific and do not illicit an allergic response.

10. The immunologically active peptide fragments of claim 1, wherein the peptides of final product are essentially free of peptides or proteins having molecular weights of about 10,000 Daltons or greater.

11. The immunologically active peptide fragments of claim 1, wherein the peptides of final product have molecular weights not more than about 2,500 Daltons.

12. The immunologically active peptide fragments of claim 1, wherein the peptides of the final product are essentially free of bacteria to avoid bacterial-induced enzymatic degradation

13. The immunologically active peptide fragments of claim 1, wherein the peptides of final product have not more than about 10% of its component peptides in a denatured state

14. The immunologically active peptide fragments of claim 1, wherein the peptides of final product contain about 0.3-0.4% proteins (nitrogen×6.38).

15. The immunologically active peptide fragments of claim 1, wherein the peptides of final product are free of coliform, Staphylococcus aureus, yeast and mould and pathogen.

16. The immunologically active peptide fragments of claim 1, wherein the peptides of final product have standard plate count (cfu/g) not more 10.

17. The immunologically active peptide fragments of claim 1, wherein the peptides of final product appear clear green liquid with slightly acidic favour

18. The immunologically active peptide fragments of claim 1, wherein the peptides of final product have shelf life of about 365 days in dry, aseptic and refrigerated environmental condition.

19. The immunologically active peptide fragments of claim 1, wherein the peptides of final product contain about 22% of proline.

20. The immunologically active peptide fragments of claim 1, wherein the peptides of final product contain high proportion of non-polar amino acids.

21. The immunologically active peptide fragments of claim 1, wherein the peptides of final product contain low percentage of glycine.

22. The immunologically active peptide fragments of claim 1, wherein the peptides of final product contain no alanine, arginine and cysteine residues.

23. The immunologically active peptide fragments of claim 1, wherein the peptides of final product contain leucine as the only N-terminal amino acid

24. The immunologically active peptide fragments of claim 1, wherein the peptides of final product contain a block of sequences of proline residues, thereby forming helices of poly-proline II type.

25. The immunologically active peptide fragments of claim 1, wherein the proline-rich polypeptides of final product are soluble at 4° C. and reversibly precipitated on warming to room temperature.

26. The immunologically active peptide fragments of claim 1, wherein the proline-rich polypeptides of final product maximally precipitate at pH 4.6 and at ionic strength above 0.6.

27. The immunologically active peptide fragments of claim 1, wherein the proline-rich polypeptides of final product have no effect of other proteins, Ca²⁺ and Zn²⁺ ions on the precipitation.

28. The immunologically active peptide fragments of claim 1, wherein the proline-rich polypeptides of final product are not an amphipathic protein.

29. A pharmaceutical formulation comprises immunologically active peptide fragments of a proline-rich polypeptide of claims 1 to 28, isolated from mammalian colostral fluid and pharmaceutically acceptable carriers.

30. The pharmaceutical formulation of claim 29, which is in the form of spray for oral administration of PRP in an effective amount so that can be held or circulated in mouth for dew seconds before swallowing.

31. The pharmaceutical formulation of claim 29, which can be directly administered in an effective amount as a teaspoon of liquid in the mouth so that it can be held in contact with oral mucosa for a few minutes and then swallowed.

32. The pharmaceutical formulation of claim 29, which can be directly administered in mouth in an effective amount, for example ¼ to 1 teaspoon.

33. The pharmaceutical formulation of claim 29, which be topically used or applied on burns and sutures, and used for skin healing conditions.

34. A method for treatment of viral and non-viral diseases or diseased conditions comprises administrating orally pharmaceutically effective amount of formulation of claim 29, containing immunologically active peptide fragments of a proline-rich polypeptide.

35. The method of treatment of claim 34, wherein the formulation can be used effectively to treat a wide range of diseases or diseased conditions such as bacterial, mycobacterial, fungal, parasitic, viral diseases or diseased conditions.

36. The method of treatment of claim 34, wherein the formulation can be administered as an oral delivery spray or directly held in the mouth for not less than 30 seconds as a teaspoon of dose to allow sorption through the mucous membrane of the mouth.

37. The method of treatment of claim 34, wherein the formulation can be administered twice a day, wherein each administration consists of five successive 1 ml sprays for adults with non-viral infections.

38. The method of treatment of claim 34, wherein the formulation can be administered twice a day, wherein each administration consists of two or three 1 ml sprays for children with non-viral infections.

39. The method of treatment of claim 34, wherein the formulation can be administered twice a day, wherein each administration consists of one 1 ml spray for infants with non-viral infections.

40. The method of treatment of claim 34, wherein the formulation can be administered every four hours, wherein each administration consists of five successive 1 ml sprays for adults with viral infections.

41. The method of treatment of claim 34, wherein the formulation can be administered every four hours, wherein each administration consists of two or three 1 ml sprays for children with viral infections.

42. The method of treatment of claim 34, wherein the formulation can be administered every four hours, wherein each administration consists of one 1 ml spray for infants with viral infections.

43. The method of treatment of claim 34, wherein the formulation can be administered through other routes that include injection, topical application, intraocular application, nebulization or atomization, and the like.

44. A method for processing mammalian colostral fluid comprises: passing a colostral fluid through at least one ion exchange column at a first pH level selected to remove large particles at a maximum fluid pressure of 30 pounds per square inch (psi); adjusting the pH of the fluid to be a value less than 5.0; and filtering the fluid with at least one filter selected from a microfilter having an initial pore size no greater than 300 nm and an ultrafilter having a pore size no greater than 20,000 Daltons.

45. The method of claim 44, further comprises adjusting a pH of a retentate trapped by said at least one filter to a value between 6.5 and 7.0; and washing antibiotics from said retentate.

46. The method of claim 44, wherein said maximum fluid pressure is less than 10 psi.

47. The method of claim 44, wherein said first pH level is in the range of 6.5 to 7.0.

48. The method of claim 44, wherein said first pH level is less than 4.0.

49. The method of claim 44, further comprising releasing said large particles from said ion exchange column with a rinse solution having a pH selected for releasing said large particles.

50. The method of claim 44, further comprising selecting said ion exchange column to comprise an anionic resin.

51. The method of claim 44, further comprising selecting said ion exchange column to comprise a cationic resin.

52. A system for processing mammalian bodily fluids comprises: at least one ion exchange column operable with a fluid pressure of less than 30 pounds per square inch (psi); and at least one filter selected from a microfilter having an initial pore size no greater than 300 nm and an ultrafilter having a pore size no greater than 20,000 Daltons, said at least one filter being operable with said fluid pressure of less then 30 psi.

53. The system of claim 52, wherein said ion exchange column and said at least one filter are operable with a fluid pressure of less than 10 psi.

54. The system of claim 52, wherein said ion exchange column removes large particles from a mammalian bodily fluid having a pH level in the range of 6.5 to 7.0.

55. The system of claim 52, wherein said ion exchange column removes large particles from a mammalian bodily fluid having a pH level of less than 4.0.

56. The system of claim 19, wherein said ion exchange column comprises an anionic resin.

57. The system of claim 19, wherein said ion exchange column comprises a cationic resin.
